(19) United States
(12) Patent Application Publication
(10) Pub. No.: US 2003/0055360 A1
(43) Pub. Date: Mar. 20, 2003

(54) Minimally invasive sensing system for measuring rigidity of anatomical matter

(76) Inventors: Matthew A. Zeleznik, Pittsburgh, PA (US); Kyle S. Lebouitz, Pittsburgh, PA (US); Michele Migliuolo, Pittsburgh, PA (US); Kaigham J. Gabriel, Pittsburgh, PA (US); Keith Rebello, Pittsburgh, PA (US); Curtis Stone, Pittsburgh, PA (US)

Correspondence Address: Finnegan, Henderson, Farabow,, Garrett & Dunner, L.L.P., 1300 I Street, N.W., Washington, DC, 20005-3315, US

(21) Appl. No.: 10/233,565

(22) Filed: Sep. 4, 2002

Related U.S. Application Data
(60) Provisional application No. 60/317,313, filed on Sep. 5, 2001.

(51) Int. Cl. A61B 5/103

(52) U.S. Cl. 600/587

(57) ABSTRACT

Apparatus are provided for determining relative and absolute properties of anatomical matter, such as biological tissue including arteries as well as other surgical and medical materials. An illustrative apparatus includes a probe for probing the properties of the anatomical matter. The probe includes a probe shaft having a sense rod, where the sense rod has a proximal end and a distal end, and where the distal end has a probe tip that is configured to contact and probe the anatomical matter. The probe also includes a sensor in mechanical communication with the sense rod, where the sensor is configured to at least measure tissue relative tissue rigidity and/or absolute tissue rigidity, including rigidity of a material on an interior surface of the tissue, a fluid flow rate in an interior of the tissue, a fluid pressure in an interior of the tissue, and/or a viscosity of a fluid in an interior of the tissue. The probe optionally includes a temperature sensor in thermal communication with the probe tip, where the thermal sensor can provide additional data, such as data related to inflammation of the tissue. Methods for probing the rigidity and other physical properties of the anatomical matter are also provided.

### CROSS-REFERENCE TO RELATED APPLICATIONS

This claims priority to U.S. Provisional Patent Application No. 60/317,313, filed Sep. 5, 2001, and U.S. patent application Ser. No. 09/626,273, filed Jul. 25, 2000, both of which are hereby incorporated by reference in their entireties herein.

### FIELD OF THE INVENTION

The present invention relates to a minimally invasive sensor system or hardness sensor for measuring properties of materials. More particularly, this invention relates to systems, sensors, and methods for measuring properties of biological tissue and other materials used in the practice of medicine or surgery, including determining the hardness, rigidity, and/or density of materials, and/or determining the flow and/or viscosity of substances in the materials, and/or determining the temperature of tissue or substance within materials.

### BACKGROUND OF THE INVENTION

Identification of calcified regions of a carotid artery during coronary bypass surgery is important for coronary bypass surgery to be effective. In order for a bypass to be most effective, the grafted vessel must be placed as close as possible to the atherosclerotic plaque, thereby allowing blood to be transported through a majority of the original vessel. One current approach to identifying the best position for the graft is accomplished by the surgeon pressing on the vessel and using tactile and visual feedback from the vessel to sense where the plaque is located. Although generally accepted, such a method relies on a qualitative assessment by the surgeon to identify the best region to perform the bypass.

Therefore, there is a need for sensing systems and methods for determining relative and/or absolute tissue rigidity and related tissue properties, including the identify and character of plaque components, inflammation, and the flow and viscosity of materials within tissue.

Further, there is a need for systems and methods for interrogating anatomical matter during minimally invasive surgery capable of converting a mechanical signal related to tissue properties into an equivalent electrical signal to allow qualitative, semi-quantitative, or quantitative data to be collected and used to locate the best position for the bypass or other surgical procedure.

### SUMMARY OF THE INVENTION

Consistent with the present invention, an apparatus is provided to determine relative tissue rigidity and/or absolute tissue rigidity, including rigidity of a material on an interior surface of the tissue, a fluid flow rate in an interior of the tissue, a fluid pressure in an interior of the tissue, and/or a viscosity of a fluid in an interior of the tissue. The apparatus includes a probe for probing the rigidity of anatomical matter. The probe includes a probe shaft having a sense rod, where the sense rod has a proximal end and a distal end, and where the distal end has a probe tip that is configured to probe anatomical matter. The probe also includes a sensor in mechanical communication with the sense rod, where the sensor is configured to measure relative tissue rigidity and/or absolute tissue rigidity, including rigidity of a material on an interior surface of the tissue, a fluid flow rate in an interior of the tissue, a fluid pressure in an interior of the tissue, and/or a viscosity of a fluid in an interior of the tissue. The probe optionally includes a temperature sensor in thermal communication with the probe tip, where the thermal sensor can provide additional data, such as data related to inflammation of the tissue, to further characterize the tissue. Methods for probing the rigidity and other tissue properties are also provided.

The invention can use micromachined, microfabricated, micromechanical, or microelectromechanical systems as sensors. These systems include devices fabricated by techniques common to or derived from the art of semiconductor processing and MEMS processing. For example, a MEMS pressure sensor can be used to convert mechanical energy to electrical energy. The electrical energy can then be converted to a signal that is correlated with the rigidity of the anatomical matter.

Consistent with this invention, apparatus and methods can include or can use, respectively, a minimally invasive tool that includes a sensor and an actuator. The minimally invasive tool can detect differences in the mechanical rigidity of anatomical matter, such as tissues, blood vessels, or tumors, in conjunction with the actuator moving the probe tip relative to the anatomical matter.

It is understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate several embodiments of the invention.

FIG. 1 is a cross-sectional view taken along the longitudinal axis of an illustrative hardness sensor consistent with the present invention.

FIG. 2 is an enlarged cross-sectional view taken along the longitudinal axis of another illustrative hardness sensor having a support tube consistent with the present invention.

FIG. 3 is another enlarged cross-sectional view taken along the longitudinal axis of yet another illustrative hardness sensor having a pressure sensor consistent with the present invention.

FIG. 4 is another enlarged cross-sectional view taken along the longitudinal axis of yet another illustrative hardness sensor having a variable capacitive sensor consistent with the present invention.

FIG. 5 is another enlarged cross-sectional view taken along the longitudinal axis of yet another illustrative hardness sensor having a displacement sensor consistent with the present invention.

FIG. 6 is another enlarged cross-sectional view taken along the longitudinal axis of yet another illustrative hardness sensor having a strain sensor consistent with the present invention.

FIG. 7 is a perspective view of an illustrative hardness sensor mounted to a robotic arm in a surgical system consistent with the present invention.

FIG. 8 is another perspective of an illustrative hardness sensor coupled with a computer controlled micropositioner actuator consistent with the present invention.

FIG. 9 is a perspective view of an illustrative hand-held device having a displacement actuator consistent with the present invention.

FIG. 10 is another perspective view of another illustrative hand-held device having an ultrasonic actuator consistent with the present invention.

FIG. 11 is a cross-sectional view of the illustrative ultrasonic actuation device of the illustrative hand-held device shown in FIG. 10, taken along line 11-11, consistent with the present invention.

FIG. 12 is a perspective view of the hand-held device shown in FIGS. 10 and 11, consistent with the present invention.

FIG. 13a is an elevational view of an illustrative hardness probe not in contact with a surface to be interrogated, consistent with the present invention.

FIG. 13b is another elevational view of an illustrative hardness probe in contact with a surface to be interrogated, consistent with the present invention.

FIG. 14 is another cross-sectional view taken along the longitudinal axis of yet another illustrative hardness sensor having a contact sensor consistent with the present invention.

FIG. 15 is another cross-sectional view taken along the longitudinal axis of still another illustrative hardness sensor having a fluid-based contact sensor consistent with the present invention.

FIG. 16 is a schematic diagram of the fluid-based contact sensing system shown in FIG. 15.

FIG. 17 is a plot showing the sensor output voltage as a function of displacement from a hardness sensor consistent with the present invention.

FIG. 18 is a plot showing the offset-corrected sensor output voltage as a function of displacement from a hardness sensor consistent with the present invention.

FIG. 19 is a plot showing the rate of change of the sensor output voltage as a function of displacement from a hardness sensor consistent with the present invention.

FIG. 20 is a plot showing the sensor output voltage change per step as a function of displacement from a hardness sensor consistent with the present invention.

FIG. 21 is a plot showing the sensor output voltage as a function of displacement from a hardness sensor for a calibration curve, a hard surface, and a soft surface, consistent with the present invention.

FIG. 22 is a plot showing for the response of a hardness sensor as a function of fluid pressure within an uncoated artery, consistent with the present invention.

FIG. 23 is a plot showing the response of a hardness sensor for an uncoated porcine artery, a porcine artery coated internally with polyurethane, and a porcine artery coated internally with cyanoacrylate as a function of fluid pressure within the respective arteries, consistent with the present invention.

### DESCRIPTION OF CERTAIN EMBODIMENTS OF THE INVENTION

Reference will now be made in detail to certain embodiments of the invention, examples of which are illustrated in the accompanying drawings. Wherever possible, the same reference numbers will be used throughout the drawings to refer to the same or like parts.

It will be understood that the term microelectromechanical systems (“MEMS”) means any microfabricated, micromachined, micromechanical, or microelectromechanical device, which includes any device fabricated wholly or partially by techniques and technologies common to or derived from the art of semiconductor processing, microchip fabrication, or MEMS processing. For example, this includes, without limitation, devices fabricated via the thin film deposition, etching, and lithographic techniques common to the field of semiconductor processing, as well as the surface and bulk micromachining techniques common to the field of MEMS processing.

It will be understood that the phrase “in mechanical communication,” when used in the context of elements of a probe, means that the elements are connected such that energy, force, and/or pressure can be transmitted between the elements. Accordingly, when a first element is in mechanical communication with a second element, the first and second elements are connected such that energy, force, and/or pressure can be transmitted between the first element and the second element. Thus, one or more intermediate elements may be used to form the mechanical communication.

The term “determine,” and grammatical variants thereof, as used herein in the context of, for example, “determine the rigidity the anatomical matter” means determining an absolute rigidity, a relative rigidity, or both. Thus, the phrase “to determine” encompasses absolute and relative measurements and characterization of the property being determined.

The terms “mechanically anchored,” “mechanically joined” and grammatical variants thereof, as used herein in the context of, for example, “a first element is mechanically anchored to a second element,” means that the first element and the second element are physically joined or connected. The connection may be, for example, formed by brazing, welding, soldering, or epoxying the two components together, or using threaded and/or fitted connections, or any combination thereof. Mechanically anchored components may also be connected together with a fluid tight seal. A fluid tight mechanical anchoring optionally may be based solely on or further include o-rings and/or press-fittings. Mechanically anchored components may also be formed integrally as a single unit.

In one embodiment, an apparatus is provided for interrogating the rigidity of anatomical matter. The apparatus can include a sense rod having a probe tip and a sensor in mechanical communication with the probe tip. The sensor can at least measure rigidity, changes in rigidity, or both, of anatomical matter interrogated with the probe tip.

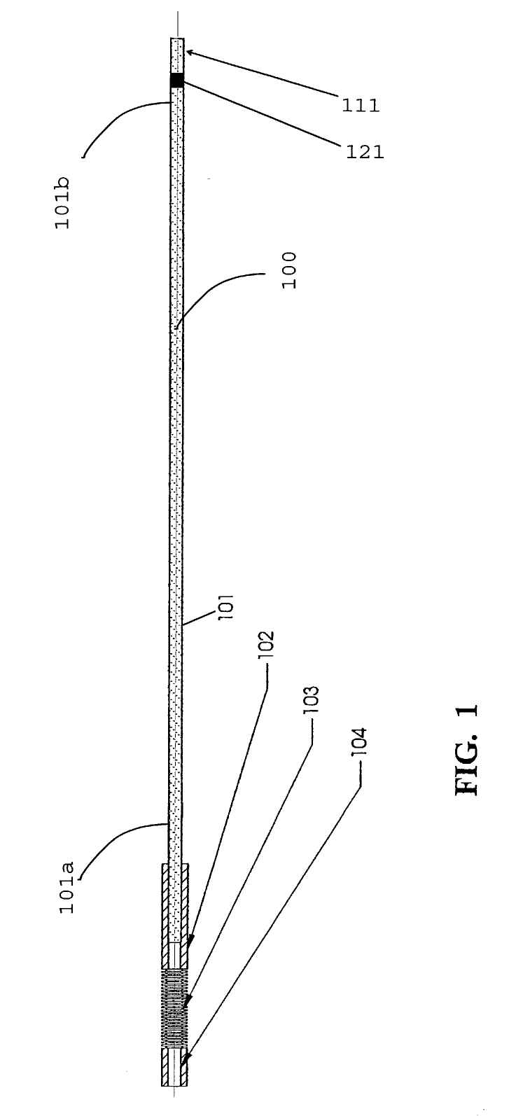

FIG. 1 schematically illustrates a probe apparatus consistent with one embodiment of the invention. The apparatus includes fixed and movable components, where the movable components are movable relative to the position of the fixed components. The movable components of the probe include sense rod 101, which has proximal end 101a and distal end 101b. Distal end 101b includes probe tip 111. Sense rod 101 is mechanically anchored to one end of miniature bellows 103 through adapter tube 102. The fixed components include adapter ring 104, which can be mechanically anchored to the other end of miniature bellows 103.

Sense rod 101 can be fabricated from a mechanically suitable material, such as stainless steel, and have a diameter and length chosen to minimize bending and buckling while having a cross-section small enough to fit into small holes or incisions used in minimally invasive surgery. For instance, sense rod 101 may have a cross section less than about ½ inch, or less than about ¼ inch. Probe tip 111 may also be dimensioned to have a tip diameter (or cross-sectional surface area) that is smaller, larger or similar in size to the anatomical matter being probed. For example, probe tip 111 may be dimensioned to have a tip diameter (or cross-sectional surface area) that is smaller, larger, or similar in size to a width of a coronary artery, such as a coronary artery in need of a bypass. A probe tip smaller in size than the portion of anatomical matter to be interrogated may be used to obtain relatively detailed point-by-point measurements, for example, in order to map changes in rigidity on a relatively small scale. A probe tip similar in size to or larger than the portion of anatomical matter to be interrogated may be used to obtain rigidity measurements averaged over the entirety of the area being interrogated.

Sense rod 101 may be longitudinally elongated between proximal end 101a and distal end 101b along longitudinal axis 100, as schematically shown in FIG. 1. The cross section of sense rod 101 perpendicular to longitudinal axis 100 may be any shape, such as circular, oval, square, or rectangular, and may be non-uniform along the length of sense rod 101. For example, proximal end 101a may have a larger or smaller cross section than distal end 101b and/or proximal end 101a may have a shape that is the same as, or different from, distal end 101b. The cross-sectional shapes and/or sizes of distal and proximal ends 101a and 101b, respectively, may also be the same as or different from any other portion of sense rod 101, such as a central portion disposed between ends 101a and 101b. Sense rod 101 need not be straight and/or colinear with respect to axis 100 along its entire length. For instance, sense rod 101 may contain one or more bends or angles anywhere along its length, provided that suitable means of maintaining mechanical communication along the length of sense rod 101 are maintained. For example, mechanical communication may be maintained by incorporating lengths of flexible or pliable material that will permit mechanical communication around bends and angles, and/or by incorporating a system of gears and related mechanical components to maintain mechanical communication between ends 101a and 101b.

Bellows 103 can be fabricated, for example, by electroforming methods. For example, electroforming methods are described in U.S. Pat. Nos. 6,036,832, 6,019,784, 6,004,447, and 5,932,076, the disclosures of which are hereby incorporated by reference herein. Bellows may also be rolled, hydroformed, welded, and/or chemically deposited.

Probe tip 111 optionally may be detachably connected to sense rod 101. For example, probe tip 111 optionally may be connected to sense rod 101 via optional connection 121, which can be a threaded and/or fitted connection that is optionally an integral part of sense rod 101. Also, a system according to the present invention may include a probe and a plurality of interchangeable probe tips of various sizes and configurations optimized for different applications and/or uses. Probe tip 111 may be formed from a material that can be sterilized and re-sterilized for medical applications. Alternatively, probe tip 111 may be disposable, and provided to customers in a pre-sterilized form and designed for single or limited use.

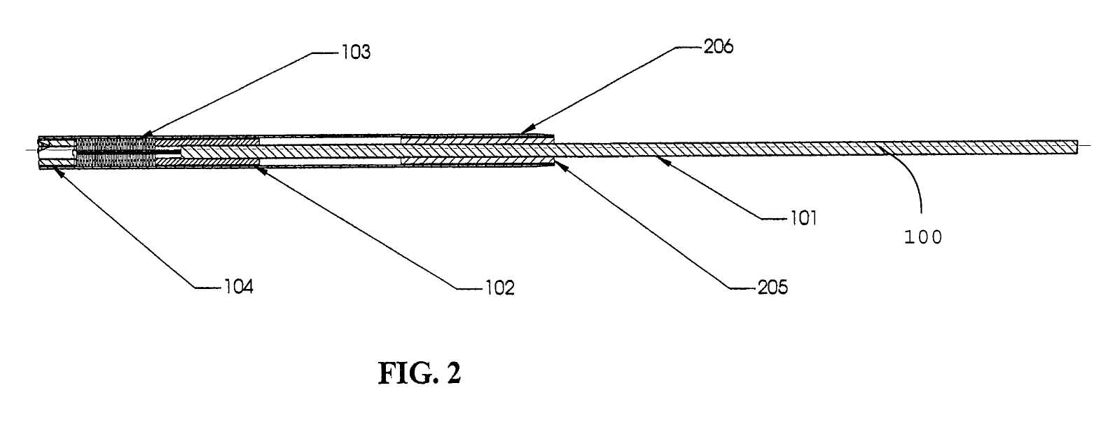

To restrict off-axis motion of sense rod 101, adapter tube 104 and bellows 103 along longitudinal axis 100, outer support tube 206 and friction minimizing tube 205 (shown in FIG. 2) can be incorporated by mechanically anchoring the end of outer support tube 206 to adapter ring 104, which in turn mechanically anchors the end of bellows 103 attached to adapter ring 104. A mechanical connection between outer support tube 206 and adapter ring 104 will allow the rest of bellows 103, adapter tube 102, and sense rod 101 to move freely along the longitudinal axis. The addition of friction minimizing tube 205, which can be made from, or internally coated with, TEFLON® or another material having a relatively low coefficient of friction, along the inner surface of outer support tube 206 allows for low-friction support of sense rod 101 as well as the reduction of the area exposed to bodily fluids during application. The inner surface of friction minimizing tube 205 can be sufficiently large such that the motion of sense rod 101 is not substantially restricted, yet sufficiently small to minimize off-axis deflections of sense rod 101.

In one embodiment, a mechanical signal related to the rigidity of the surface to be measured is generated by placing probe tip 111 of sense rod 101 against the surface of the anatomical matter to be measured, such that a surface of probe tip 111 perpendicular to longitudinal axis 100 is in contact with the surface of the anatomical matter, while simultaneously displacing outer support tube 206 along longitudinal axis 100 and towards the anatomical matter. The displacement of outer support tube 206 and consequently the end of bellows 103 attached to adapter ring 104 causes a reaction force to be generated by any anatomical matter sensed with a magnitude representative of the rigidity of the anatomical matter. The reaction force is then transferred from sense rod 101 to bellows 103 causing bellows 103 to compress. The amount of compression is a function of the ratio of the reaction force to the spring constant of bellows 103. The compressed bellows, which represents the mechanical rigidity of the probed anatomical matter, can be used to thus create a number of measurable signals. Using different sensing techniques, the signals created by the compressed bellows can be obtained and those signals can be further used to determine the absolute and/or relative rigidity of the anatomical matter. This determination can be based on, for example, an amplitude of the signal, a change in the signal as a function of probe displacement, a rate of change of the signal, a comparison of the signal to one or more calibration signals or standards, or any combination thereof.

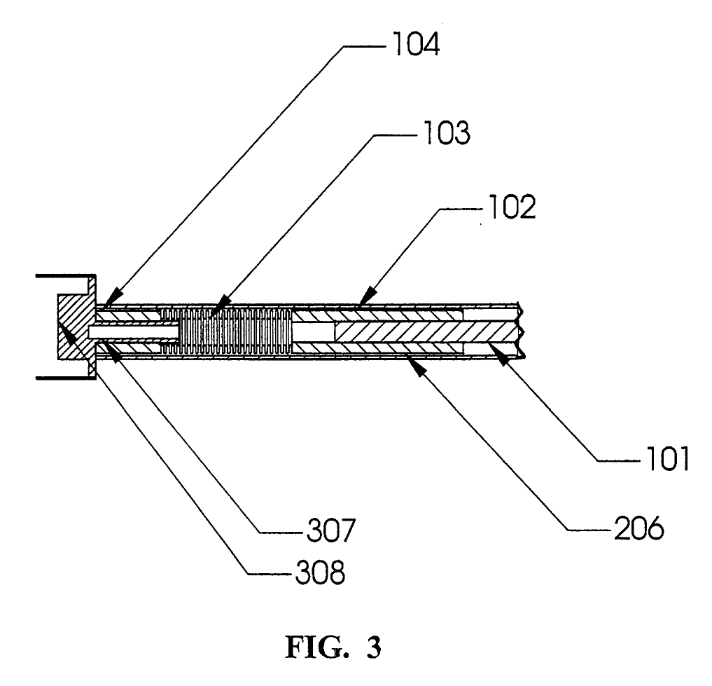

A pressure sensor can be used, as illustrated in FIG. 3, for sensing the response of the bellows from the reaction force described above. In this approach, for example, a leak-tight seal between sense rod 101, adapter tube 102, miniature bellows 103, adapter ring 104, and inlet port 307 of pressure sensor 308 forms a sealed chamber. The seal can be accomplished, for example, by mechanically anchoring these components together. It is possible to use vacuum fittings to attach any of these components together, however this may be at the expense of added device diameter. Utilization of MEMS-based pressure sensor 308, such as, for example, the SM5852 series by Silicon Microstructures Inc, of Fremont, Calif., allows for a small footprint, although a larger, non-MEMS based pressure sensor can also be used consistent with this invention.

The sealed chamber that includes on at least one side bellows 103 contains a fixed amount of air or other compressible fluid or material. In response to the reaction force produced by placing probe tip 111 against the anatomical matter to be probed and by displacing outer support tube 206 in the direction of probe tip 111, bellows 103 will be compressed. Although not wishing to be bound by any particular theory, bellows 103 is believed to be compressed by a ratio of the reaction force to the spring constant. The compression of the bellows in turn creates a smaller volume for the air (or other compressible fluid or material), which adds resistance to further compression. From Boyle's Law it follows that the pressure inside of the sealed chamber increases proportionally with decreasing volume. The compression of bellows 103 thus creates an increased pressure inside of the sealed chamber that can then be sensed using pressure sensor 308 with a suitable sensitivity.

Pressure sensor 308 should be within, or in fluid communication with, the sealed chamber such that it is sensitive to the absolute, relative, and/or differential pressure in the sealed chamber. A wide range of pressure sensor sensitivities can be used since it is possible to provide a wide range of spring constants and sealed chamber volumes, which partially determine a suitable pressure range and sensitivity for the pressure sensor, using any of the process by which bellows 103 are created. The compressible fluid or material may also be selected to determine a suitable pressure range and sensitivity.

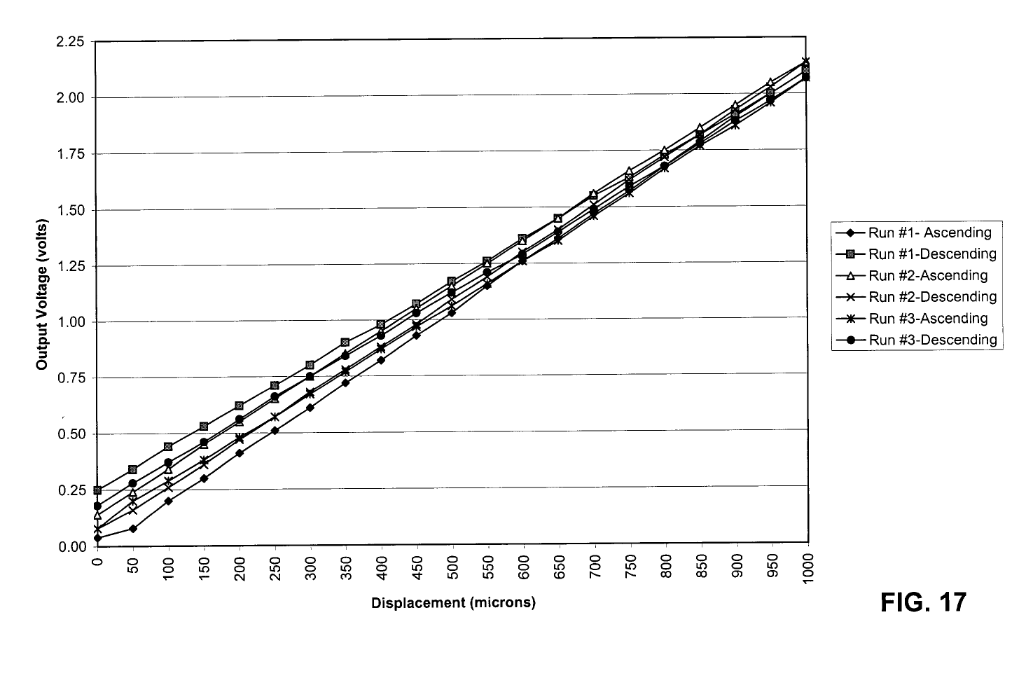

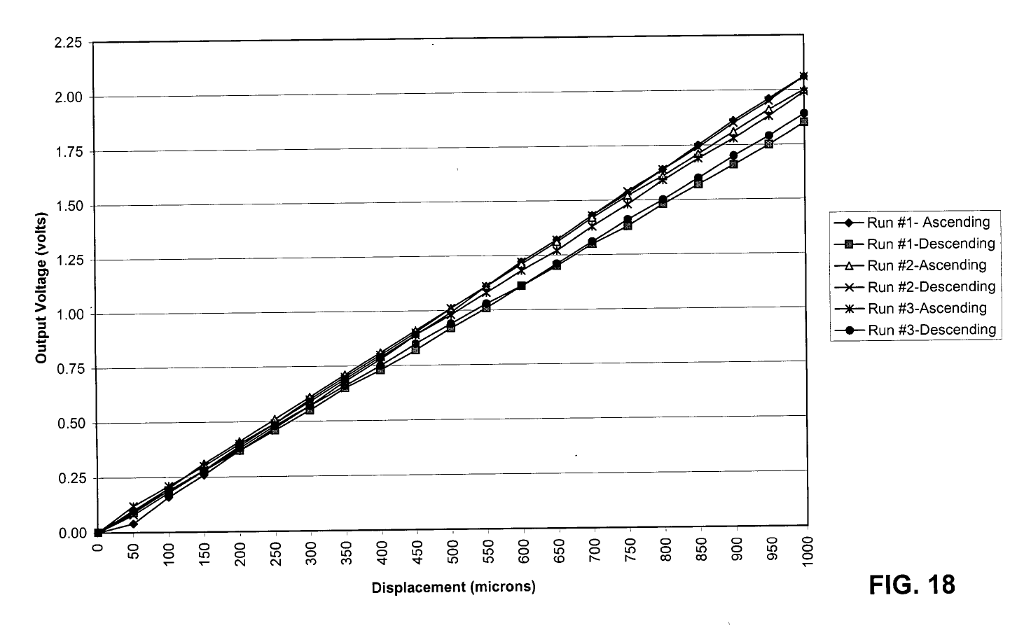

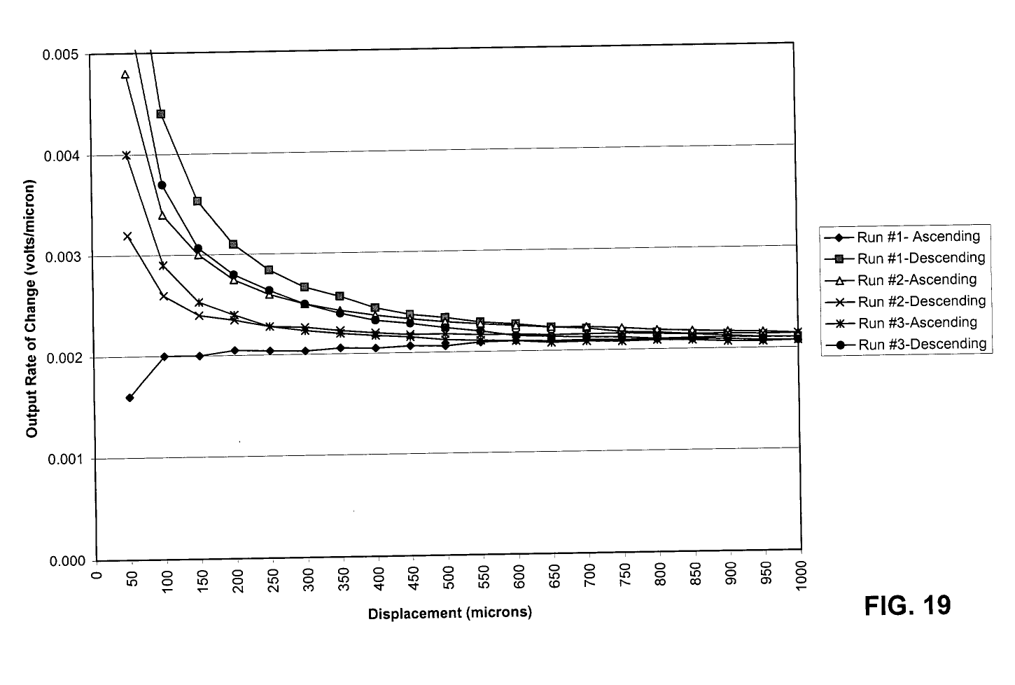

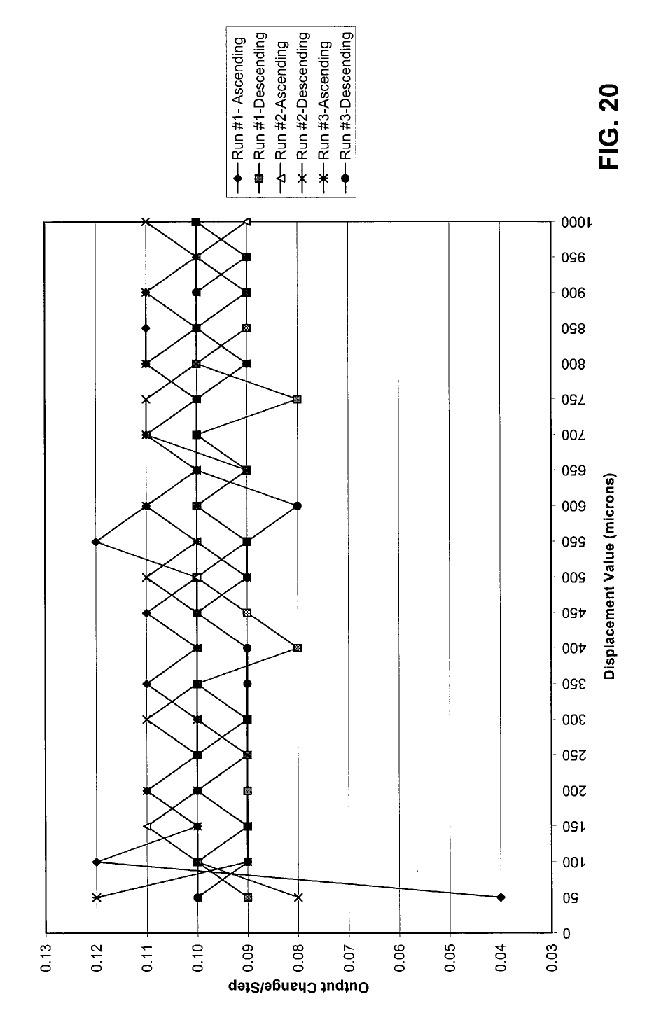

FIGS. 17-20 show results obtained from the above configuration using a 0.30 psi piezoresistive MEMS pressure sensor, specifically the SM5852-003-G-3-L sensor from Silicon Microstructures of Fremont, Calif. Measurements were taken by clamping the outer support tube of a hardness sensor consistent with the present invention and using a micropositioner to displace sense rod 101 along its longitudinal axis towards (ascending) and away from (descending) the clamped outer support tube, thereby mimicking the operation of the device to probe anatomical matter. A linear response over a 1 mm displacement of the micropositioner was observed indicating that the device operates according to the model.

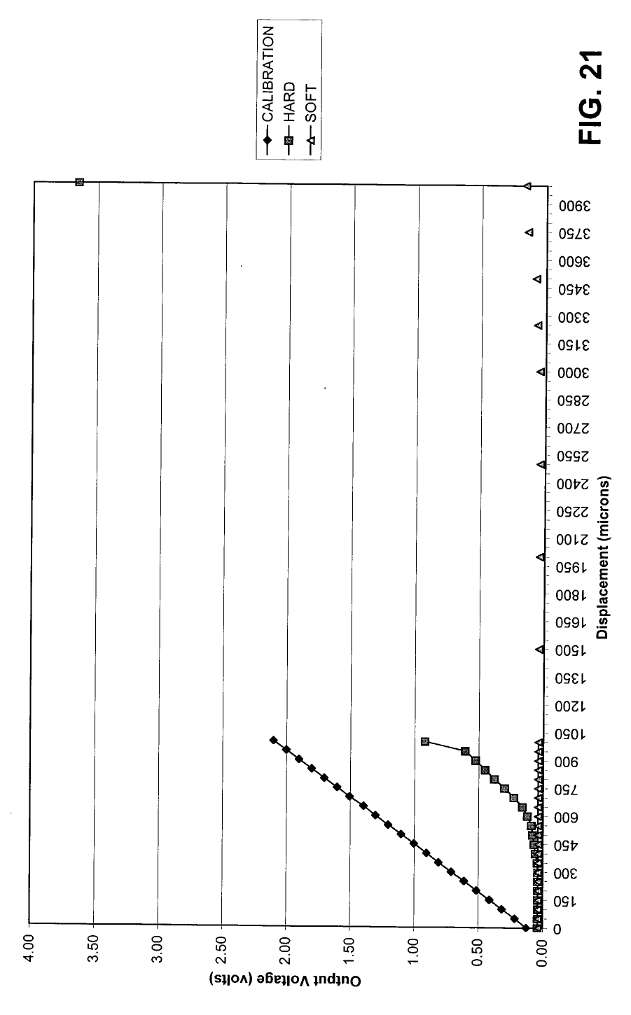

Measurements were conducted on non-biological materials to simulate the response obtained when probing a healthy artery, which is relatively soft, and a calcified artery, with is relatively hard. The measurements entailed contacting the hardness sensor with a relatively soft surface, foam rubber, and a relatively hard surface, STYROFOAM®, to simulate the properties of a healthy artery and a calcified artery, respectively. The hardness sensor was then displaced towards the respective surface. As shown in FIG. 21, significant differences in the response of the device, as indicated by the sensor output voltage, between the hard and soft surface measurements and the sensor calibration data were observed.

In addition to identifying regions of hardness and/or plaque it can be important to identify and characterize the components of the plaque. This can assist in distinguishing stable, calcified plaque from soft, vulnerable lesions that are filled with fat and inflammatory cells and encased by only a thin, fibrous cap. For example, the assessment of atherosclerosis is important to gauge the risk of heart attack and also stroke, which can result from plaque rupture in the carotid arteries and aorta. The composition of plaque is made up of calcium, lipid, the fibrous cap and thrombis. By determining the makeup of the plaque, it may be possible to predict which plaques are stable and which are vulnerable to rupture. The coronary arteries do not have pain fibers, and both swelling and redness can have many other causes, therefore heat remains a valid sign of inflammation in the coronaries. Inflammation plays a key role in the destabilization of plaque. The collection of information related to the identity and character of components of plaque, including information related to inflammation, may provide valuable insight into vessel wall pathology.

Additionally, the measurement of flow and viscosity of materials within tissue can be a valuable tool to the determine, for example, the viability of anastamosis, reattached vessels, during replantation of limbs, incorporation of vascular grafts, transplant surgery, and other applications.

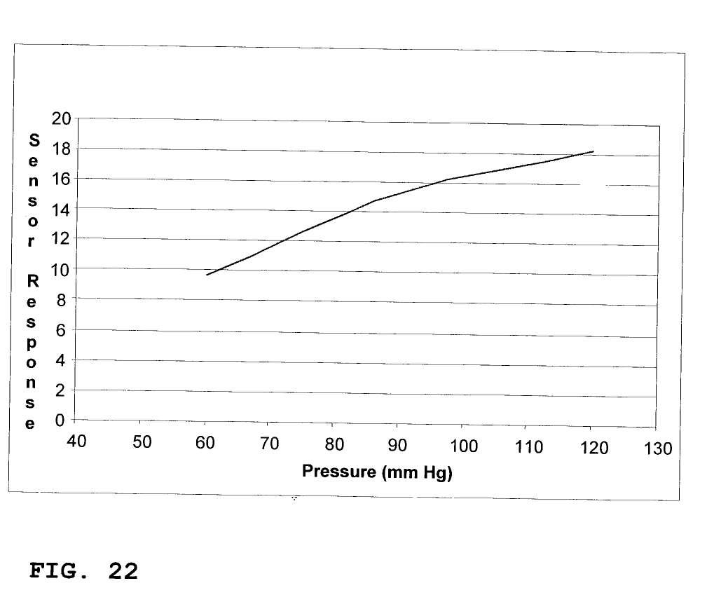

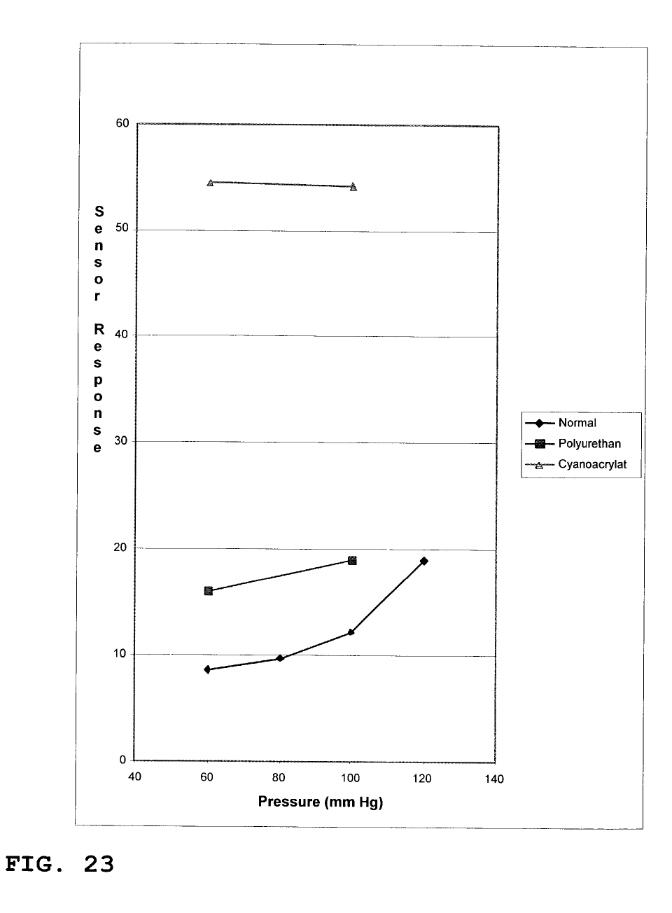

Measurements were conducted with a hardness sensor consistent with present invention on biological tissue having different internal hardness and fluid pressures. More specifically, measurements performed on six porcine hearts by inserting the left anterior descending (LAD) coronary artery of each specimen into a closed loop perfusion circuit. The circuit contained a pump which was used to vary the fluid pressure in the artery in order to evaluate the instrument's response to different fluid pressures. Additionally, different levels of artery hardness were investigated by leaving the interior artery wall uncoated or natural (lowest hardness) and by coating the interior artery wall with polyurethane (medium hardness) or cyanoacrylate (severe hardness). The instrument is able to detect changes in artery pressure of an uncoated artery as shown in FIG. 22 as a change in the response of the sensor as a function of fluid pressure within the uncoated artery. The instrument is also able to detect differences in artery wall hardness as shown in FIG. 23, also shown as a change in response of the sensor as function of pressure for each of uncoated or normal artery (line with diamond symbols), polyurethane coated artery (line with square symbols), and cyanoacrylate coated artery (line with triangular symbols). As shown in FIG. 23, the response at any given pressure differed for each of the type of tissue measured.

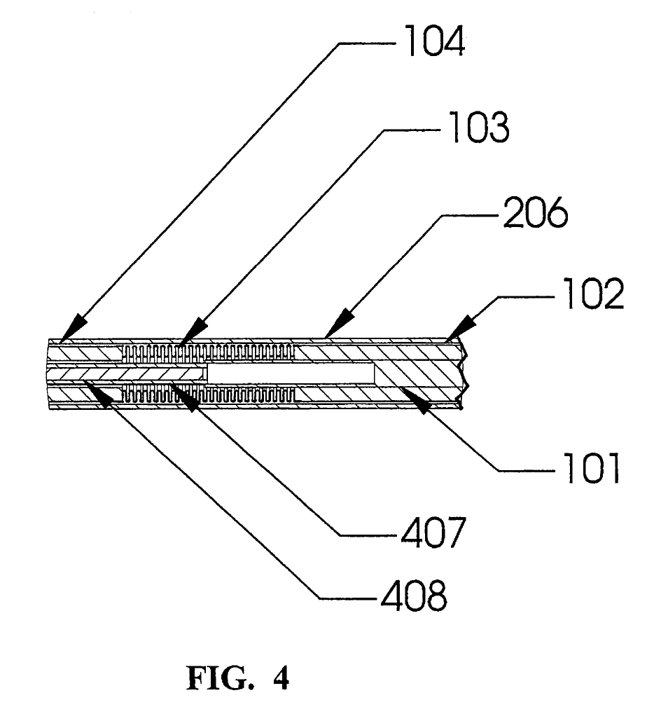

A capacitance sensor placed in mechanical communication with the probe tip can be used to produce a signal correlated with the rigidity of the probed surface. For example, as shown in FIG. 4, a variable capacitor design can be used to detect deflection of bellows 103 and yield a change in capacitance of a coaxial capacitor related to the deflection of bellows 103. This change can then be measured and correlated with the rigidity of the matter being probed. Outer electrode 101 of the coaxial capacitor can be formed, for example, by the protrusion of sense rod 101 into bellows 103. To provide sense rod 101 with dual functionality, the core of the sense rod may be machined to form a tube at the end of sense rod 101 attached to adapter tube 102. Inner electrode 408 and dielectric tube 407 may be attached to the inside of adapter ring 104. To increase the capacitance of the device, dielectric tube 407 may be made out of a material that exhibits a high dielectric constant, such as alumina. The device can include mechanical joints between sense rod/outer electrode 101 and adapter tube 102, adapter tube 102 and miniature bellows 103, miniature bellows 103 and adapter ring 104, adapter ring 104 and dielectric tube 407, and dielectric tube 407 and inner electrode 408. According to one embodiment, the joint between dielectric tube 407 and inner electrode 408 can be force fit.

In response to a reaction force during operation, bellows 103 deflects allowing outer electrode 101 to overlap dielectric tube 407 and inner electrode 408 by substantially the same distance as the deflection of bellows 103. The overall capacitance at rest is given by the capacitance of a coaxial capacitor with a length of zero. Once deflected, the length of the capacitor increases by the displacement of the bellows, thus increasing the capacitance and creating a capacitive signal. A coaxial capacitor design, as described, requires that the inner diameter of outer electrode 101 be larger than the outer diameter of dielectric tube 407 to ensure freedom of motion under an applied reaction force. To ensure proper alignment, inner electrode 408 should be shorter than dielectric tube 407 such that, at rest, outer electrode 101 overlaps dielectric tube 407 while minimizing the rest capacitance. Optimization of this device can be achieved based on the radii of the electrodes and dielectrics, as well as the overlap distances.

Inner electrode 408 also may be in the form of, for example, a plate and the end surface of the distal end of sense rod 101 may be in the form of a second plate. Thus, a variable parallel plate capacitor which operates in a fashion similar to above is formed.

The displacement of the bellows can be used to generate a signal using, for example, a variable reluctance transducer (“VRT”), including a linear variable reluctance transducer (“LVRT”) or a differential variable reluctance transducer (“DVRT”). Any other form of displacement sensor, including, for example, optical techniques, such as laser interferometry can also be used.

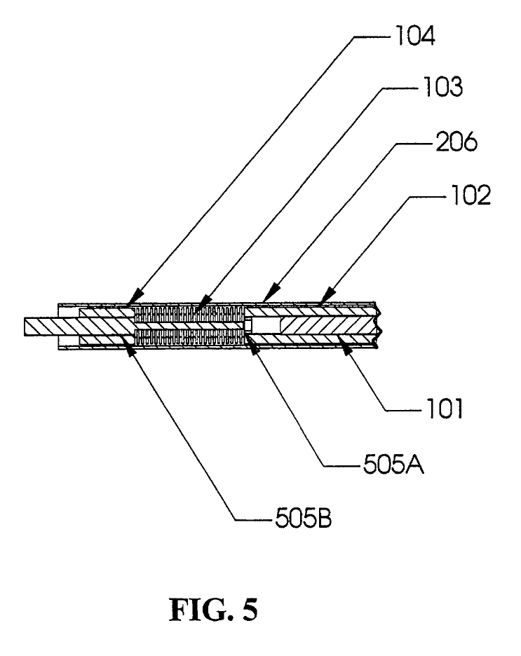

FIG. 5 shows one illustrative embodiment of a system that uses a VRT displacement sensor. In this system, sense rod 101 can be mechanically anchored to adapter tube 102, which in turn can be mechanically anchored to miniature bellows 103. At the end of bellows 103 nearest adapter tube 102, the magnetic core of the VRT 505a can be mechanically anchored to bellows 103. According to one embodiment, the anchoring may use, for example, an epoxy or similar bond. The opposite end of bellows 103 is mechanically anchored to adapter ring 104. The VRT sensor 505b is then anchored to the inner surface of adapter ring 104.

During operation, the reaction force from the anatomical matter being probed causes sense rod 101 to compress the free end of miniature bellows 103 and displace magnetic core 505a of the VRT along its longitudinal axis. As magnetic core 505a is inserted into the bore of VRT sensor 505b in response to the compression of bellows 103, a Hall effect sensor within the VRT can be used to measure the distance traveled by magnetic core 505a, resulting in an output voltage from the VRT indicative of the magnitude of the displacement. Suitable VRTs include the line of DVRT sensors from Microstrain, Inc. of Willison, Vt. and those described in U.S. Pat. No. 4,813,435, which is hereby incorporated herein by reference.

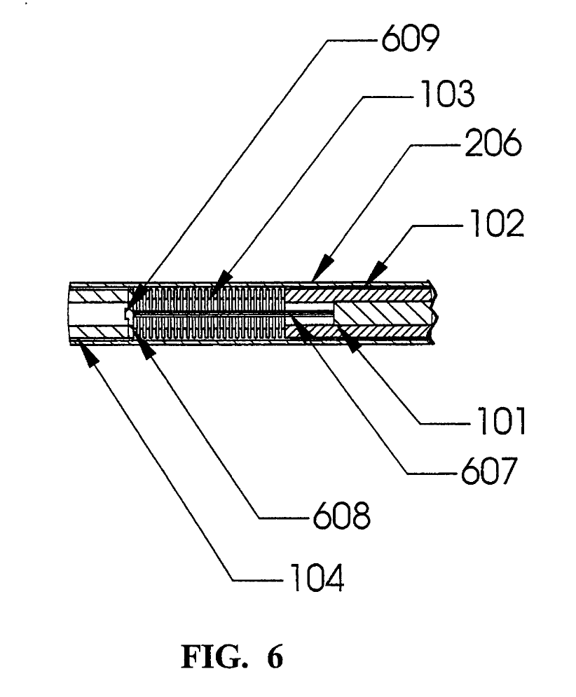

According to one embodiment, the probe may include a device for sensing the strain induced by a boss on a membrane. For example, FIG. 6 shows thin membrane 608 mechanically joined to bellows 103 and adapter ring 104. In turn, adapter ring 104 can be mechanically anchored to outer support tube 206. Micromachined strain gauge 609, which is mechanically anchored to or integrated within the backside of membrane 608, can be used as the sensing element. Machined boss 607, referred to herein as an actuation tip, is machined from or mechanically anchored to the distal end of sense rod 101, which can be mechanically anchored to adapter tube 102. A mechanical joint between adapter tube 102 and miniature bellows 103 completes the assembly, as shown in FIG. 6.

In response to the reaction force from the probed anatomical matter, sense rod 101 and actuation tip 607 compress bellows 103, thereby bringing actuation tip 607 into contact with membrane 608. Further displacement of actuation tip 607 causes membrane 608 to deflect in response to the reaction force, thereby inducing a strain in membrane 608. The strain can be measured according to the resistance of a piezoresistive strain gauge 609, and can optionally be converted into an output voltage through a suitable circuit such as a Wheatstone Bridge configuration. The magnitude of the resistance, or output voltage if applicable, thus corresponds to the magnitude of the reaction force.

Optimization of this device can be performed by selecting, in relation to the types and rigidities of anatomical matters tested, the diameter of actuation tip 607, and the distance between membrane 608 and actuation tip 607. The thickness, diameter, and material used to form membrane 608, the sensitivity and design of strain gauge 609, as well as the spring constant for bellows 103 can also be selected as desired. Strain gauges may be formed by, for example, the methods disclosed in U.S. Pat. No. 6,341,528, which is hereby incorporated by reference herein. Commercially available strain gauges include the ESB-020-500 and ESB-020-350 silicon strain gauge from Entran Devices, Inc. of Fairfield, N.J. and the SA-XX-008CL-120 metal foil strain gauge from Vishay Measurements Group of Raleigh, N.C.

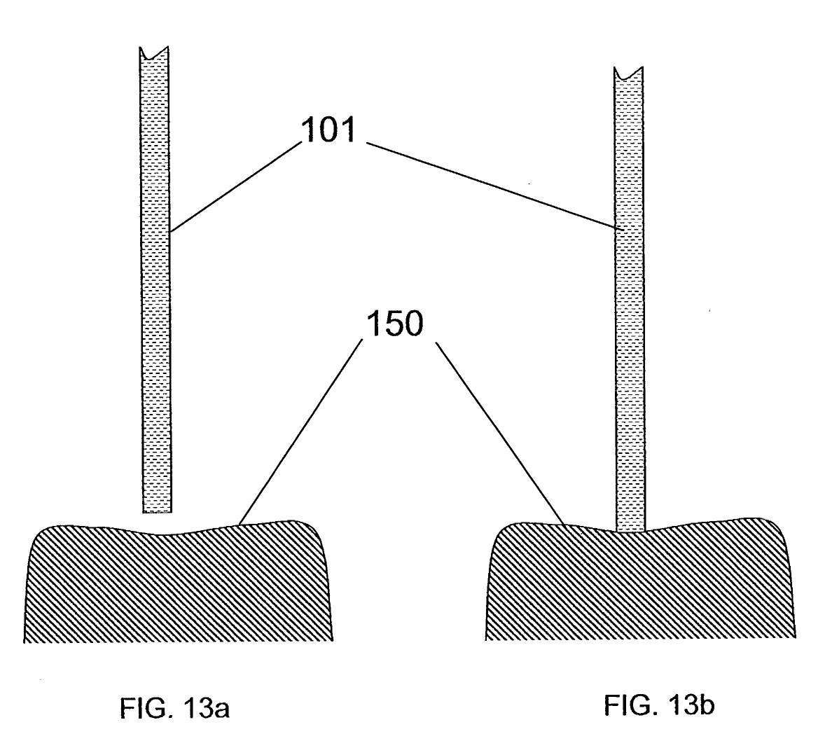

One of the challenges in effectively using a hardness sensor is knowing when the sense rod is in contact with the tissue. This challenge is depicted in FIG. 13a, where sense rod 101 is not in contact with tissue 150. In contrast, FIG. 13b shows sense rod 101 in contact with tissue 150. Conventional methods for determining whether sense rod 101 is in contact with tissue 150 requires an operator to observe the contact, often from an inconvenient or visually restricted angle. This can be further complicated when the sense rod is brought near the tissue because fluids and other obstructions in the line-of-sight can prevent accurate observation. Further, even under the best circumstances, it can be difficult to visually determine whether one object is a few hundred micrometers separated from another object or whether those objects are in contact. For measurements that are done on a displacement scale of between a few hundred micrometers and a few millimeters, not knowing an exact position of contact can substantially degrade measurements.

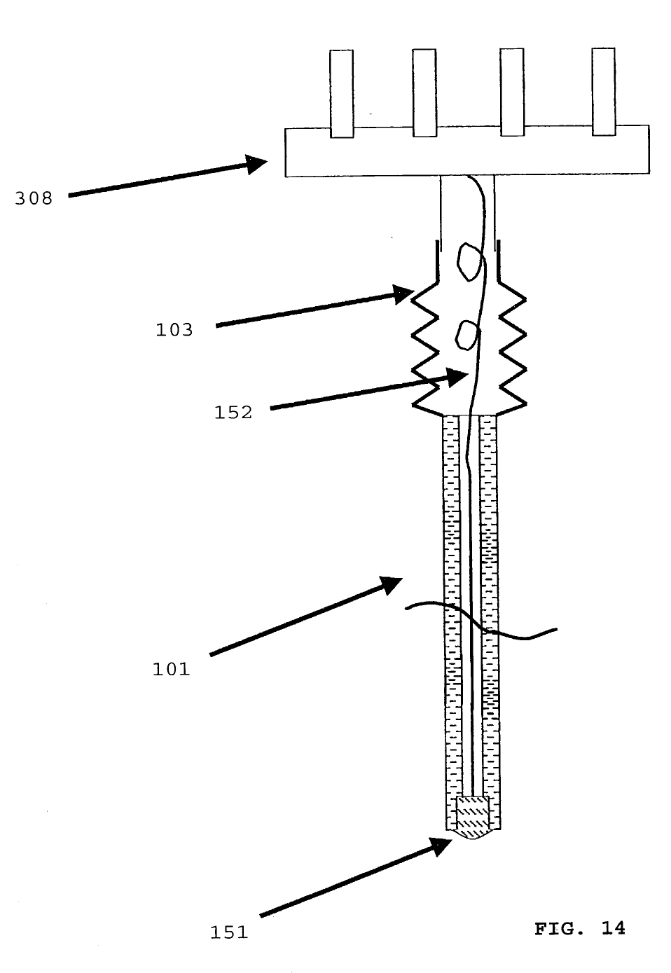

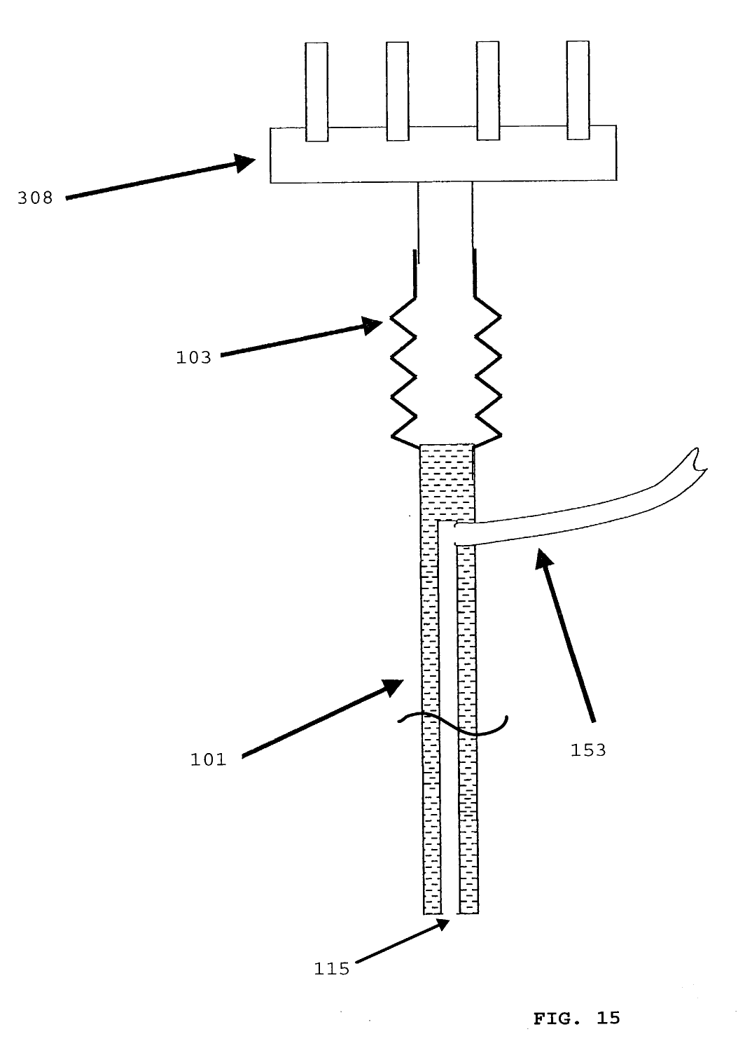

To address this issue, a probe consistent with this invention can include a point contact sensor located at or in communication with the probe tip. For example, FIG. 14 shows a probe that includes contact sensor 151, which is mounted in or integrated with the distal end of sense rod 101. This contact sensor can be any sensor capable of measuring the contact of the probe tip to the surface. For example, contact sensor 151 can be an ultrasonic sensor, a set of contacts under a thin membrane known as a membrane switch, a fluid pressure sensor, a temperature-based sensor, or any combination thereof. It will be appreciated that any other sensor or suitable configuration capable of sensing contact with solid anatomical matter can also be used consistent with this invention.

An ultrasonic sensor, when used as contact sensor 151, can use shifts in frequency or phase of ultrasonic signals to identify the proximity of the probe tip to tissue 150. Alternatively or additionally, the delay between ultrasonic pulse-echo signals can be used to measure distance or proximity, similar in concept to sonar ranging. Further shifts in frequency, phase, and/or delay will occur as a force is applied between the sensor and the tissue. These shifts may be used to provide additional verification of contact and/or tissue rigidity to the operation of the hardness sensor.

Suitable ultrasonic sensors can be made from piezoelectric materials or films, such as PVDF (Polyvinylidene Fluoride) or PZT (Lead Zirconate Titanate). For example, a piece of PVDF film can be attached to a glass substrate, where a circuit for the ultrasonic element is formed by an electrical lead (such as an electrical lead formed by photolithography) on the substrate connected to the lower side of the PVDF film and an electrical connection to the upper side of the PVDF film, which is metalized. An ultrasonic element can also be a membrane-style device, which emits ultrasonic sound waves by converting an electrical signal into a mechanical deflection or vibration of a membrane, and which detects ultrasonic radiation by the reverse process (i.e. converts a mechanical deflection/vibration in the membrane into an electrical signal).

A membrane switch closes when pressed against tissue. A sensing method using a membrane switch as contact sensor 151 relies on the membrane of the membrane sensor being sufficiently thin and flexible, specifically more compliant than the tissue being measured. Thus, by pressing against tissue, the switch, which typically includes miniature contacts produced by microfabrication, can close without significant deformation of the tissue.

A temperature-based sensor can be used as contact sensor 151. It works by measuring the temperature at the tip of the sensor. As the tip gets closer to the tissue, the temperature typically rises to a temperature that is near a normal tissue temperature (e.g., 98.6° Fahrenheit). Alternatively, a heater can be placed near the sensing part of temperature based contact sensor 151 to warm the tip of sensor 151 above the normal temperature, so that, when sensor 151 contacts the tissue, the sensor cools towards normal tissue temperature. Instead of or in addition to serving as a contact sensor, a temperature sensor in thermal communication with the probe tip may be used to determine and/or compare the localized temperature at different locations in or on biological tissue, for example, to determine the temperature and hence the degree of liquidity of plaque inside an artery wall. This temperature data could augment the rigidity data and further assist the doctor in making decisions. A suitable temperature sensor might be a thermocouple. Commercially available temperature sensors include the IT-23 from Physitemp Instruments, Inc. of Clifton, N.J. A temperature sensor in thermal communication with the probe tip, such as temperature based contact sensor 151, may also provide additional information regarding the properties of the biological tissue, such as its temperature, which can be indicative of inflammation. A temperature sensor in thermal communication with the probe tip, such as temperature based contact sensor 151, may also provide a temperature compensation signal for other measurements conducted with the probe.

According to another embodiment, sense rod 101, bellows 103, and pressure sensor 308 can form one vacuum tight assembly, as shown in FIG. 14. To maintain a vacuum tight seal, contact sensor 151 can be mechanically anchored to sense rod 101 with a vacuum tight seal. For example, a sufficient seal can be formed using o-rings and/or press-fittings.

Connection to contact sensor 151 can be achieved by using internal wire 152 that connects to a contact inside of a package containing contact sensor 151 allowing the system to maintain the vacuum seal without external wires. Wire 152 can be looped so as to minimize stiffness increases to the hardness sensor system that could occur, for example, from tension if wire 152 is stretched or compressed. Thinner wires may also be used to minimize stiffness. Wire 152 can be, for example, 30 gauge or thinner; however, thicker wires are not excluded.

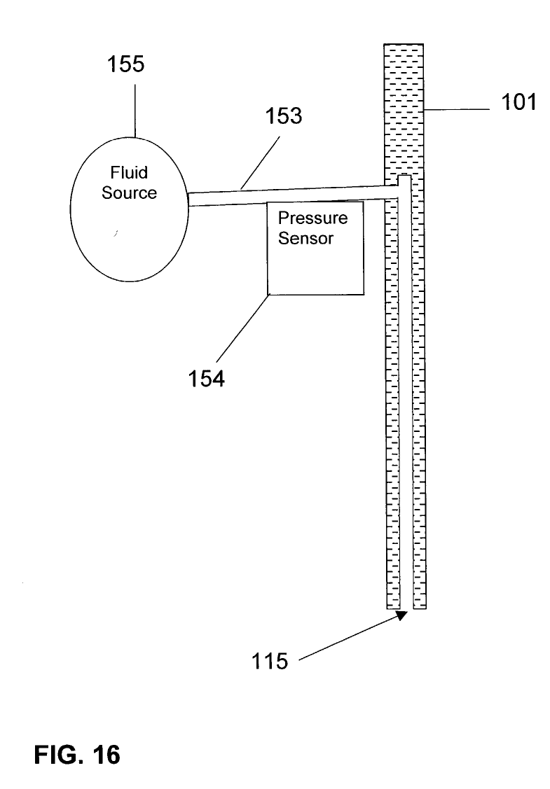

The point contact sensor may include a fluid pressure sensor. For example, FIG. 15 shows fluid tube 153 connected to a partial bore through the side of sense rod 101 such that it is in fluidic communication with fluid outlet 115. As shown in FIG. 16, fluid tube 153 can be in fluid communication with fluid source 155, which can be, for example, a slightly pressurized container of liquid or gas. Further, fluid tube 153 can be in fluid communication with pressure sensor 154 that senses the pressure within fluid tube 153.

When sense rod 101 is pressed against tissue, fluid outlet 115 of sense rod 101 seals, causing the pressure to rise in fluid tube 153. Partial sealing of fluid outlet 115 may also occur, and will also yield increased pressure in fluid tube 153. Therefore, by monitoring the reading from pressure sensor 154, tissue contact can be determined.

The fluid used in the sensor may be a saline solution, but other fluids, including water or gas, such as air, can also be used. Air, however, poses a slight danger because a rupture in the vessel during the use of the hardness probe could inject air bubbles, possibly causing an embolism. In any case, the pressures and flow rates used can be extremely small. For example, flow rates can be on the order of microliters per minute and pressures can be below 1 pound per square inch. When contact sensor 151 is a fluid sensor, the fluid emitted from the probe tip can also be used for irrigation and, if fluid tube 153 is connected to a negative pressure source, suction.

A system for hardness and contact sensing can operate in a manner that is substantially the same as for a probe without a contact sensor. However, a system for hardness and contact sensing can also include monitoring and/or determining contact of the probe tip with the surface, at least partially based on a signal or change in signal from the contact sensor.

A method of operating a probe consistent with the present invention can include applying a repeatable force or displacement to outer support tube 206 of the probe apparatus, which induces a reaction force from the anatomical matter sensed. By applying a predetermined displacement to outer support tube 206, the magnitude of the reaction force will not be affected by the device itself; it will depend primarily on the properties of the matter which are being observed. Variations introduced by a non-repeatable displacement of outer support tube 206 will ultimately lead to a signal that is dependent on both the properties of the anatomical matter as well as the applied deflection. This multi-dependence convolutes the signals and making it difficult to distinguish between the effects of each component of the measured signal.

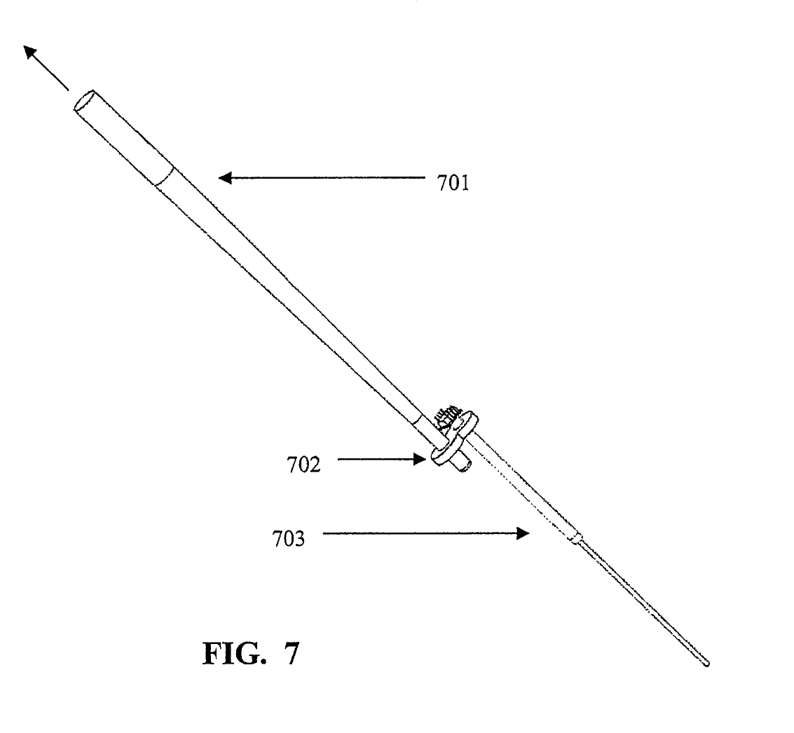

An actuating system for producing a repeatable actuation can be an end effecter or component in a robotic surgical system, such as products sold under the trademark ZEUS®, available from Computer Motion of Goleta, Calif., or the DA VINCI SURGICAL SYSTEM® available from Intuitive Surgical of Mountain View, Calif. In these systems, a surgeon operates on a patient via a computer-controlled robot with interchangeable tools at the end of the robotic arm allowing for performing different types of surgical procedures. The hardness sensor, consistent with this invention, can be placed on the end of the robotic arm, as shown for example, in FIG. 7. In FIG. 7, minimally invasive hardness sensor 703 described herein can be mounted to robotic arm 701 of a surgical system through custom machined holder 702. In one embodiment, sensor holder 702 does not allow for any undesired motion along the longitudinal axis of the apparatus. Because these systems are capable of highly accurate motions, a repeatable displacement can be applied to the outer support tube yielding a true measurement of the properties of the sensed matter.

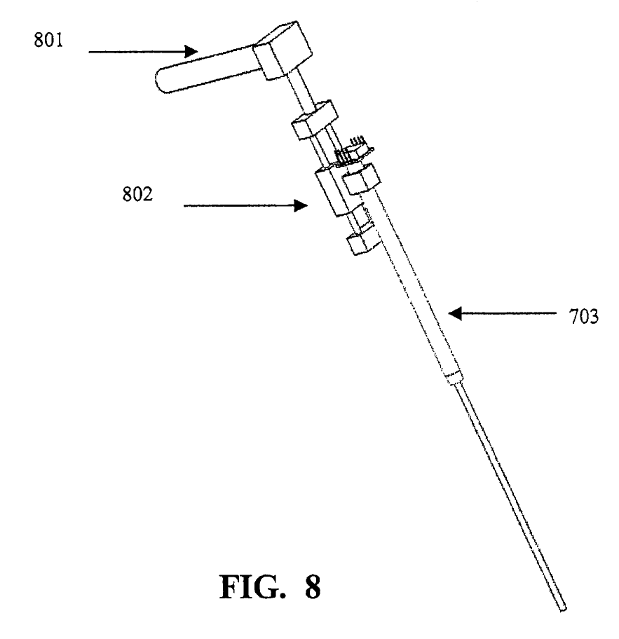

A computer controlled micropositioner, such as the NT53-674 positioner and the NT54-705 controller by Edmunds Optics™, of Barrington, N.J., can be used to hold sensor 703, as shown in FIG. 8. Computer-controlled micropositioner 802 can be mounted to a support system (e.g., system 801). The support system can be a simple laboratory stand mounted to a fixed surface, or a more intricate or complex mounting assembly. Sensor 703 can be attached to micropositioner 802 through a customized holder similar to sensor holder 702 as shown in FIG. 7. As with the robotic actuation system, displacement of sensor 703 is precisely controlled allowing for an accurate measurement of the anatomical matter. With such a system, it is possible to create a custom actuation and readout system with a virtual instrument using currently available software, such as LABVIEW®, a software program available from National Instruments of Austin, Tex., thus allowing the end user to precisely control operation of the system.

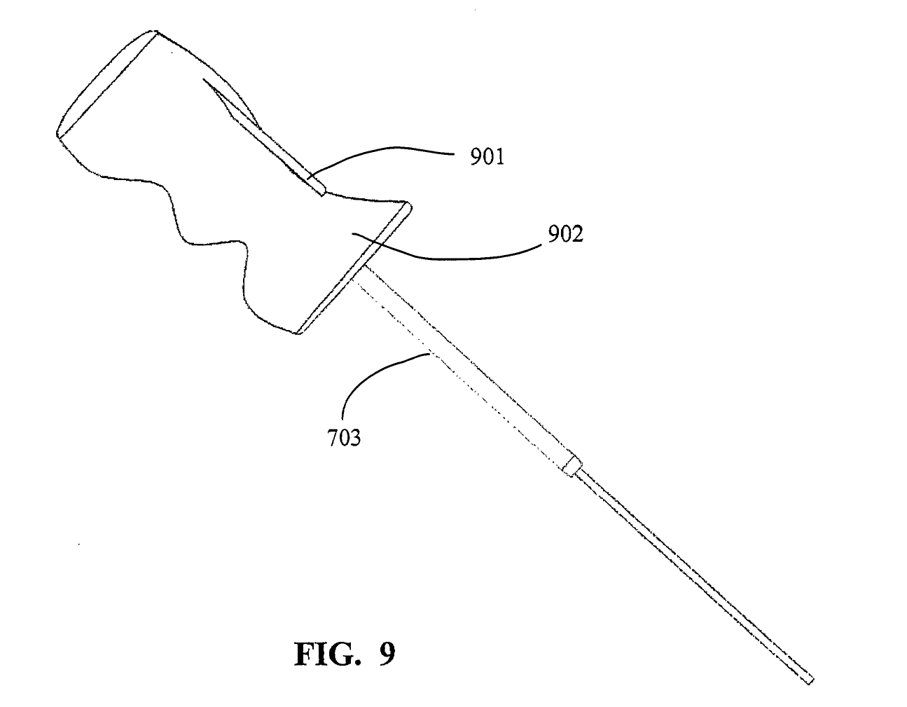

A hand-held device consistent with this invention is capable of applying a repeatable displacement of the outer support tube of the sensor with respect to the matter to be sensed. FIG. 9 depicts a hand-held controller mechanically anchored to probe 703. Upon depression of trigger 901, the outer support tube of probe 703 would be displaced relative to handle 902 by an actuator, such as a spring loaded actuator or other mechanical or electromechanical actuator. The location of the actuator, which is in mechanical communication with both handle 902 and probe 703, can be external to or internally within handle 902. In this embodiment, the deflection of the support tube relative to the handle is extremely repeatable. This approach, however, requires the surgeon to hold the handle in a steady position relative to the anatomical matter being sensed.

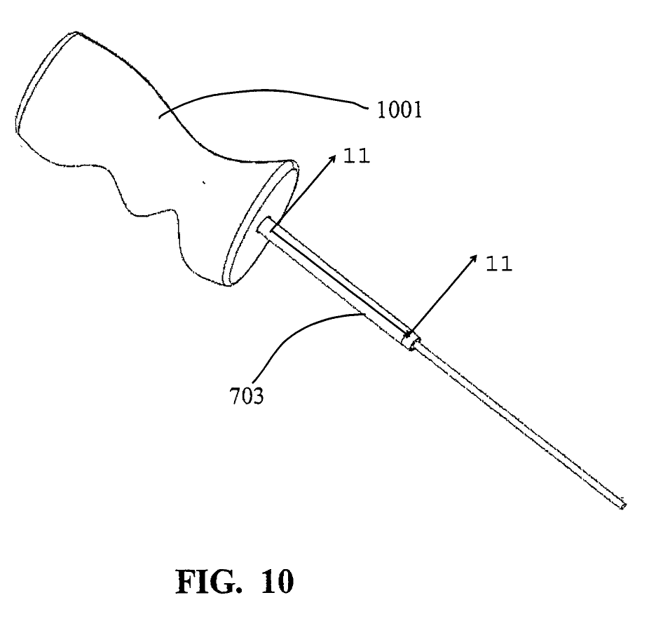

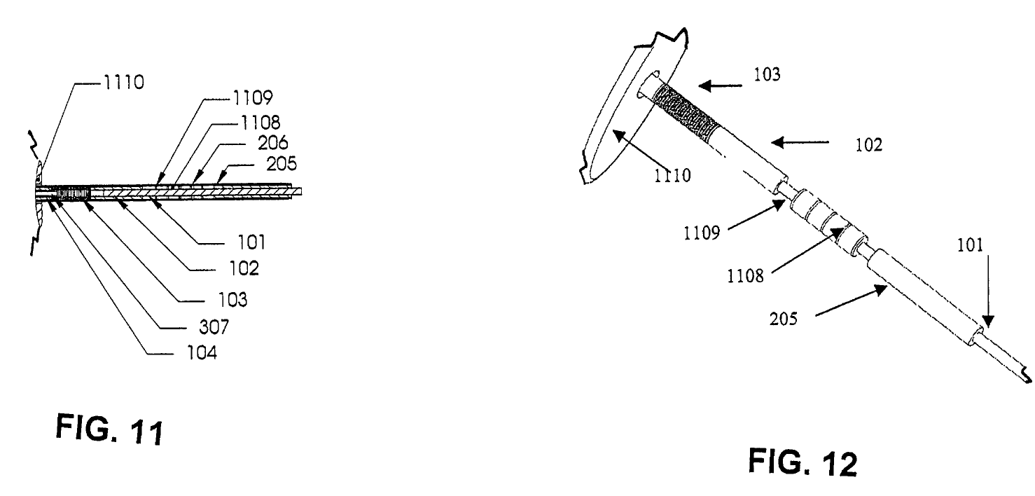

A hand-held device consistent with this invention can include at least one ultrasonic transducer in the sensing device. For example, FIG. 10 shows the general appearance of one such device with probe 703 mounted to handle 1001. Actuation, such as ultrasonic actuation, is achieved by incorporating at least one piezoelectric element 1108 and electrodes 1109 for electrical actuation of piezoelectric element 1108 along the longitudinal axis of the sensor as depicted, for example, in FIGS. 11 and 12. There is a first mechanical joint between sense rod 104 and the inner faces of piezoelectric elements 1108 and electrodes 1109, which can be, for example, an epoxy joint. There is a second mechanical joint between outer support tube 206 and piezoelectric elements 1108 and electrodes 1109 can be realized in a similar fashion. The sense rod 101 can be connected to a lower portion 1110 of the handle 1001 by a mechanical joint between the support tube 206 and the lower portion 1110 of the handle 1001, or can be connected via another other fixed element of probe 703. Sense rod 101 is thereby mechanically coupled with outer support tube 206 through piezoelectric elements 1108. The rest of the assembly is similar to the assembly already described with respect to FIGS. 1 and 2.

By driving piezoelectric elements 1108 at or near their resonant frequency, bellows 103 compresses and retracts at the same frequency, generating a signal with a set frequency and amplitude modulation. It will be appreciated that piezoelectric elements 1108 can also be oscillated at other than their resonant frequency or pulsed in a single shot mode. Using the pressure sensing device as an example, compression of the bellows due to the ultrasonic actuators causes an increase in the pressure while extension of the bellows due to the ultrasonic actuators returning to their rest state causes the pressure to return to the normal state. When the hand-held device is brought into contact with the anatomical matter to be interrogated, a coupling between the anatomical matter and the tip causes a change in the output frequency, phase, and/or amplitude of the signal generated by the pressure sensor. The mechanical rigidity of the anatomical matter being sensed can be determined using the change in frequency, phase, and/or amplitude as an indicator.

The resonant frequency, oscillation phase, oscillation amplitude, electrical resistance, and/or electrical current of the piezoelectric element itself may be monitored as a measure of the rigidity or other properties of the anatomical matter being sensed. Thus, the actuator can also function as the sensor in mechanical communication with the probe tip and a separate sensor is not necessarily required.

As opposed to the hand-held deflection actuator shown in FIG. 9, the oscillating or pulse actuated device would allow for a surgeon to merely place the tip of the probe against the matter being measured. While gross variations in the surgeon's positioning of the device with respect to the matter to be measured will cause degradation of the signal produced by the matter itself, minor variations of the position of device will be much less significant than with the hand-held displacement actuated device. Signals can be averaged across multiple oscillation or pulse cycles to further reduce effects of placement and/or the increase single to noise ratio.

It will be appreciated that a probe sensitive to the mechanical properties of anatomical matter, such as the rigidity, can be used in a variety of applications in the medical field. For example, such a probe can be used to monitor pressure exerted on a beating heart during beating heart procedures by stabilization systems, such as the OCTOPUS 3 TISSUE STABILIZER® from Medtronic. The probe may include additional sensors, such as a temperature sensor for making temperature measurements used for temperature dependent calculations and procedures.

A variety of additional materials, shapes, and sensors that are not expressly listed can be used consistent with this invention. Thus, design variations that include the general sensor and actuation systems described herein above are within the scope of the invention.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A probe for probing rigidity of anatomical matter, the probe comprising:
a probe shaft comprising a sense rod, wherein the sense rod has a proximal end and a distal end, wherein the distal end comprises a probe tip configured to contact the anatomical matter; and
a sensor in mechanical communication with the sense rod, wherein the sensor can at least make a measurement chosen from a group consisting of a measurement of the rigidity of the anatomical matter, a measurement of a change in the rigidity of the anatomical matter, a measurement of a rigidity of a material on an interior surface of the anatomical matter, a fluid flow rate in an interior of the anatomical matter, a fluid pressure in an interior of the anatomical matter, a viscosity of a fluid in an interior of the anatomical matter, and any combination of thereof.

2. The probe according to claim 1, wherein the distal end of the sense rod can fit into an incision used in minimally invasive surgery.

3. The probe according to claim 1, wherein the probe shaft is elongated and has sufficient longitudinal rigidity to minimize or eliminate bending or buckling during probing.

4. The probe according to claim 1, wherein the sensor is located in the probe tip.

5. The probe according to claim 1, wherein the sensor is not located in the probe tip.

6. The probe according to claim 1, wherein the sensor comprises a microelectromechanical system.

7. The probe according to claim 6, wherein the microelectromechanical system comprises a sensor selected from a group consisting of a pressure sensor, a capacitive sensor, a displacement sensor, a strain sensor, and any combination thereof.

8. The probe according to claim 1, wherein the probe further comprises a temperature sensor in thermal communication with the probe tip.

9. The probe according to claim 1, wherein the sensor is a microelectromechanical pressure sensor.

10. The probe according to claim 6, wherein the probe further comprises:
an adapter tube connected to the distal end of the sense rod;
a bellows connected to the adapter tube; and
an adaptor ring connected to the bellows.

11. The probe according to claim 10, wherein the probe further comprises an outer support tube having a low-friction interior surface that at least partially extends over the proximal end of the sense rod, wherein the outer support tube is connected to the adaptor ring and has an interior diameter small enough to minimize off-axis deflection of the sense rod and large enough to not restrict motion of the sense rod.

12. The probe according to claim 10, wherein the probe can make a bellows measurement selected from a group consisting of a displacement measurement of the bellows, a compression measurement of the bellows, a decompression measurement of the bellows, and any combination thereof.

13. The probe according to claim 10, wherein the probe comprises a sealed chamber defined on at least one side by the bellows, and a pressure in the seal chamber is changed by compression of the bellow, decompression of the bellows, or both; and
wherein the sensor can make a pressure measurement selected from a group consisting of a measurement of the pressure in the sealed chamber, a measurement of a change of the pressure in the sealed chamber, and any combination thereof.

14. The probe according to claim 10, wherein the sensor comprises a capacitance sensor that can detect a deflection of the bellows.

15. The probe according to claim 10, wherein the sensor comprises a displacement sensor configured to detect a displacement of the bellows.

16. The probe according to claim 10, wherein the sensor comprises a strain sensor that can make a bellows measurement selected from a group consisting of a bellows compression measurement, a bellows decompression measurement, and any combination thereof.

17. The probe according to claim 1, wherein the probe further comprises an actuator in mechanical communication with the probe, and wherein the actuator can actuate a position of the probe, the sense rod, or any combination thereof.

18. The probe according to claim 17, wherein the actuator comprises an actuator chosen from a group consisting of a piezoelectric actuator, a robotic surgical system, a pneumatic actuator, an inductively driven actuator, a magnetic actuator, an electromagnetic actuator, a micropositioner, and any combination thereof.

19. The probe according to claim 17, wherein the actuator can make an actuation chosen from a group consisting of an oscillation of the position of the sense rod, a pulsing of the position of the sense rod, and any combination thereof.

20. The probe according to claim 19, wherein the sensor comprises a sensor that can make a measurement of an element, wherein the element is chosen from a group consisting of the actuator, the sense rod, and any combination thereof, and wherein the measurement is chosen from a group consisting of a measurement of an oscillation frequency, a measurement of an oscillation amplitude, a measurement of an oscillation phase, a measurement of pulse-echo delay, a measurement of pulse-echo amplitude, a measurement of pulse-echo waveform shape, a change in any foregoing measurement, and any combination thereof.

21. The probe according to claim 19, wherein the sensor at least can make a measurement of the actuator, wherein the measurement is chosen from a group consisting of an oscillation frequency, an oscillation amplitude, an oscillation phase, a pulse-echo delay, a pulse-echo amplitude, an electrical resistance, an electrical capacitance, an electrical inductance, an electrical current, an electrical voltage, a magnetic field of the actuator, any change in the foregoing, and any combination thereof.

22. The probe according to claim 1, further comprising a point contact sensor in mechanical communication with the probe tip.

23. The probe according to claim 22, wherein the point contact sensor comprises a sensor selected from a group consisting of an ultrasonic sensor, a membrane switch, a fluid pressure sensor, a temperature based sensor, and any combination thereof.

24. The probe according to claim 1, wherein the probe tip is detachable from the sense rod.

25. The probe according to claim 1, wherein the probe tip can be substantially sterilized for medical applications.

26. A method for probing rigidity of anatomical matter, comprising:
contacting the anatomical matter with a probe comprising
a probe shaft comprising a sense rod, wherein the sense rod has a proximal end and a distal end, wherein the distal end comprises a probe tip configured to contact the anatomical matter, and
a sensor in mechanical communication with the sense rod, wherein the sensor can make a measurement chosen from a measurement of the rigidity of the anatomical matter, a measurement of a change in the rigidity of the anatomical matter, a measurement of a rigidity of a material on an interior surface of the anatomical matter, a fluid flow rate in an interior of the anatomical matter, a fluid pressure in an interior of the anatomical matter, a viscosity of a fluid in an interior of the anatomical matter, and any combination of thereof,
wherein the contacting comprises contacting the anatomical matter with the probe tip;
displacing the probe relative to the surface; and
determining the rigidity of the anatomical matter based on at least a signal generated from the sensor.

27. A method according to claim 26, wherein the determining comprising making a signal analysis chosen from a group consisting of an analysis of an amplitude of the signal, an analysis of a change in the signal as a function of probe displacement, an analysis of a rate of change of the signal, an analysis of the signal relative to a calibration signal, and any combination thereof.

28. A method according to claim 26, wherein the probe further comprises a temperature sensor and the signal from the sensor in mechanical communication with the sense rod is temperature compensated.

29. A method for probing an interior surface of anatomical matter, comprising:
contacting the anatomical matter with a probe comprising
a probe shaft comprising a sense rod, wherein the sense rod has a proximal end and a distal end, wherein the distal end comprises a probe tip configured to contact the anatomical matter,
a first sensor in mechanical communication with the sense rod, wherein the sensor can make a measurement chosen from a measurement of the rigidity of the anatomical matter, a measurement of a change in the rigidity of the anatomical matter, a measurement of a rigidity of a material on an interior surface of the anatomical matter, a fluid flow rate in an interior of the anatomical matter, a fluid pressure in an interior of the anatomical matter, a viscosity of a fluid in an interior of the anatomical matter, and any combination of thereof, and
a temperature sensor in thermal communication with the probe tip,
wherein the contacting comprises contacting the anatomical matter with the probe tip;
determining information related to the interior surface of the anatomical matter based on at least a signal generated from the first sensor; and
determining a temperature of the anatomical matter based on at least a signal generated from the temperature sensor.

30. The method according to claim 29, wherein the anatomical matter is an artery, and wherein the information related to the interior surface of the artery comprises information related to plaque components on the interior surface of the artery based on a signal generated from the first sensor.

31. The method according to claim 29, wherein the anatomical matter is an artery, and wherein the method comprises determining information related to inflammation of the artery based on at least the determining the temperature of the artery.
